(19) United States
(12) Patent Application Publication
(10) Pub. No.: US 2022/0175558 A1
(43) Pub. Date: Jun. 9, 2022

(54) DEVICE FOR CONTROLLING A ROBOTIC SYSTEM FOR ASSISTING THE MOBILITY OF A USER

(71) Applicants: Centre National de la Recherche Scientifique, PARIS (FR); INSERM - Institut National de la Santé et de la Recherche Médicale, PARIS (FR); SORBONNE UNIVERSITÉ, PARIS (FR)

(72) Inventors: Nathanaël JARRASSÉ, PARIS (FR); Mathilde LEGRAND, Zürich (CH); Guillaume MOREL, PARIS (FR)

(21) Appl. No.: 17/440,748

(22) PCT Filed: Mar. 24, 2020
(86) PCT No.: PCT/EP2020/058203

§ 371 Date: Sep. 17, 2021

(30) Foreign Application Priority Data
Mar. 25, 2019 (FR) FR1903103

(51) Int. Cl. A61F 2/70; B25J 9/00; A61F 2/58; A61H 1/02

(52) U.S. Cl. CPC A61F 2/70; B25J 9/0006; A61H 2201/1207; A61H 1/0277; A61H 2230/625; A61F 2/582

(57) ABSTRACT

The present invention concerns a device for controlling a robotic system for assisting the mobility of a user, said robotic system comprising at least one active mobility assistance element capable of assisting a given mobility action of the user, characterized in that the device comprises a detection system capable of detecting a compensatory movement of the user associated with the mobility action, said compensatory movement being a movement made by a user who is disabled, or able-bodied but locally and/or temporarily constrained, in order to perform at least part of the mobility action, and which at least partially substitutes the normal movement an unconstrained, able-bodied user would make in order to perform this mobility action, and a control system capable of controlling the at least one active element when the compensatory movement is detected.

The present invention relates generally to a device for controlling a robotic system for assisting the mobility of a user, and in particular for assisting the mobility of the upper limbs of a user. It also relates to a method for controlling a robotic mobility assistance system implementing the device.

In addition to rehabilitation exoskeletons which replay preprogrammed movements, various devices are known for controlling a robotic system for assisting the mobility of a user, in particular for the upper limbs.

A keyboard/mouse/joystick control system is thus known, which has the disadvantage of constraining the remaining functional capacities or those of the functioning limb of the user; a myoelectric control (by electromyograms); a control by the measurement of cerebral activity (for example with an electroencephalogram (EEG) measurement); a control by the movements of the face or the mouth; a simple proportional control between the movement of a movable joint and the movement of a robotic joint; a control with a movement of the feet; or a control based on joint synergies.

Documents WO 2015/140353 A2, EP 1 260 201 A1 and CN 105 456 000 B describe active mobility assistance elements for the lower limbs. In these documents, a coupling is made, but it is a case of synergy: it is the forward movements of the torso that trigger walking. Tilting the torso is not a compensation for walking (lifting the foot would be a compensatory movement); it is a synergistic anticipatory movement. More precisely, tilting the trunk forward is a movement (sometimes almost imperceptible) that everyone does when they start to walk, even when they have no mobility problem. A compensatory movement for walking is only used when the leg cannot function normally. In this case, the person tries to lift his foot and then walk by another means than with his leg. He would for example make a movement of the hips to lift his foot, accompanied by a rotation of the trunk to bring it forward (one may imagine oneself trying to walk without a crutch with a knee brace).

In the document WO 2015/106278 A2, the movements of the body or the arms are also not compensatory movements; they are synergies that the subject must learn (correlations).

A first major drawback of these devices, with the exception of the control being based on joint synergies, is that they impose an artificial control law on the user. This requires a significant learning time, up to several weeks for myoelectric control and/or control based on cerebral activity, and the difficulty of control increases rapidly with the number of joints to be controlled. In addition, most of these devices are based on a control in the joint space, i.e., each joint is controlled individually and often sequentially, one after the other. The cognitive load to perform a gesture is thus significant because the user has to break down the movement into sub-movements for each joint, which nobody does naturally.

The present invention aims to remedy these drawbacks.

The subject matter of the invention is thus a device for controlling a robotic system for assisting the mobility of a user, said robotic system comprising at least one active mobility assistance element capable of assisting a given mobility action of the user.

The device according to the invention comprises a detection system capable of detecting a compensatory movement of the user associated with said mobility action, said compensatory movement being a movement carried out by a user who is disabled, or able-bodied but locally and/or temporarily constrained, in order to perform at least part of the mobility action, and which at least partially substitutes the normal movement an unconstrained, able-bodied user would make in order to perform this mobility action, and a control system capable of controlling the at least one active element when the compensatory movement is detected.

A compensatory movement, or compensation movement, may be defined as a movement made by a user who is disabled (having a disability reducing part of his mobility) or able-bodied but locally and/or temporarily constrained, in order to perform at least a part of the mobility action, and which at least partially substitutes the normal movement an unconstrained, able-bodied user would make in order to perform this mobility action. The disabled user thus uses the functional parts of his body to compensate for the disabled, missing, deficient or constrained parts, and for example at least to initiate the mobility action.

The idea of the invention arises from the observation that, when a user is assisted by a robotic system for assisting mobility, if said robotic system is not in the geometric configuration desired to perform a task and if said user has no direct means for changing said configuration, said user will naturally tend to generate a compensatory movement, as if the robotic system were “disabled.” This observation also applies, at times, when the direct means of control exist but represent too great a cognitive or physical cost.

Thus, according to the invention, to perform a given mobility action (for example a given movement or a set of given movements), the mobility action is associated with a predetermined compensatory movement, and an active mobility assistance element is controlled as soon as the compensatory movement is detected. The active element is not controlled directly as a function of the mobility action, but by the detection of a compensation movement associated with the mobility action. Thus, the control occurs naturally, decreasing the cognitive load currently required to control the active element, and reducing the learning time to master the control.

The device according to the invention thus utilizes the body's natural reaction to a decrease in mobility. The user does not have to learn any imposed artificial control law. The control of the active element is even more natural since it takes place in the space of the mobility action (space of the task), and not in the joint space.

The invention also makes it possible to reduce the compensatory movements necessary to perform a mobility action by transferring the mobility of compensatory proximal joints (for example mobilized to compensate for the absence of an amputated limb) to robotic distal joints.

The active element may be an element for assisting the mobility of the user's upper limb. The active element may be a robotic arm prosthesis or an assisting arm exoskeleton. The mobility action may thus be to reach a target, for example using the arm, and the compensatory movement may be an inclination of the user's torso.

The active element may also be an element for assisting the mobility of a lower limb of the user.

The detection system may be adapted to detect a difference between a reference position and a position of the user, for example using one or more sensors associated with the user, and to detect a compensatory movement when said deviation reaches a predetermined deviation.

The reference position may be generally defined as being a comfortable position, without postural compensation, that the user would naturally adopt to perform the mobility action if he had control of all his joints.

In one embodiment, said at least one active mobility assistance element may be an active element intended to be secured to the user.

Said at least one active mobility assistance element may thus be a motorized joint, intended for example to control a user's defective or absent joint or an additional/supernumerary robotic joint. The active element may thus be a robotic arm prosthesis (for example a prosthetic elbow joint), for a user with an amputation of an upper limb, or even an assisting arm exoskeleton, for a user in a situation of paralysis of the extremities or muscle weakness, in a home environment.

In another embodiment, said at least one active mobility assistance element may be an active element external to the user.

Said at least one active mobility assistance element may in this case be an active element for controlling a movement of a target of the user, for example an active element for controlling a movement of an object that the user wishes to reach or an object on or with which he is working.

The control system may also be capable of introducing a correction of the compensatory movement and in particular, once the compensatory movement has been detected, the correction of the compensatory movement which may be carried out simultaneously and/or after controlling the at least one active mobility assistance element.

The subject matter of the invention is also an assistance assembly for the mobility of a user.

The assembly according to the invention comprises a previously described device and a robotic system controlled by said device.

The subject matter of the invention is also a method for controlling a robotic system for assisting the mobility of a user.

The method according to the invention implements a device described above.

The method may include the steps of:
determining a mobility action of a user,
associating said mobility action with a compensatory movement of the user,
when said compensatory movement is detected, controlling at least said active mobility assistance element, in particular so as to perform the mobility action.

Other advantages and features of the present invention will become clear from the following description, given by way of non-limiting example, and made with reference to the appended figures:

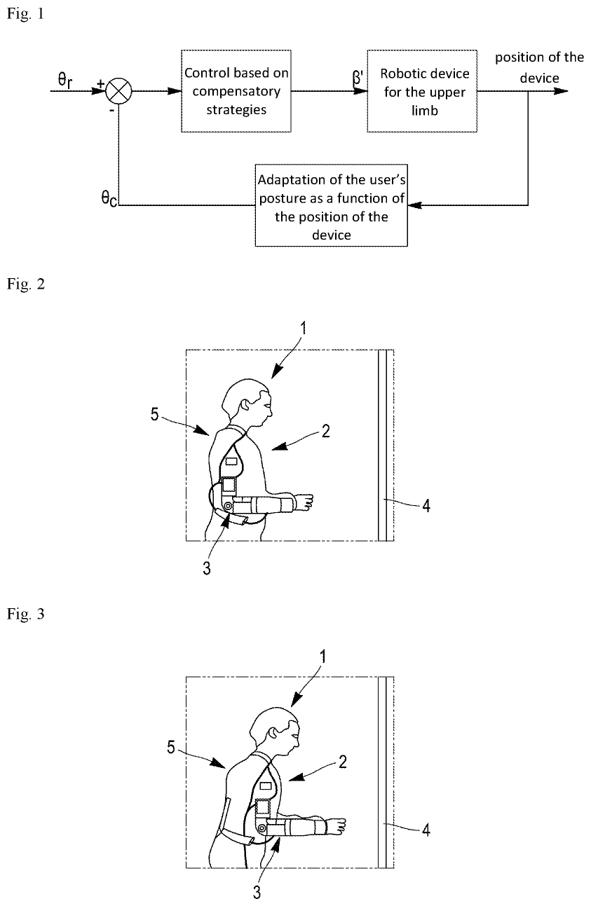

FIG. 1 is a block diagram illustrating the overall operation of a device for controlling a robotic mobility assistance system according to the invention,

FIG. 2 to 4 schematically illustrate different positions of a user performing a mobility action using the control device according to the invention,

FIG. 5 is a graph illustrating the progression over time of the inclination angle of the trunk and the flexion angle of the elbow of the user in FIG. 2 to 4, according to a first embodiment,

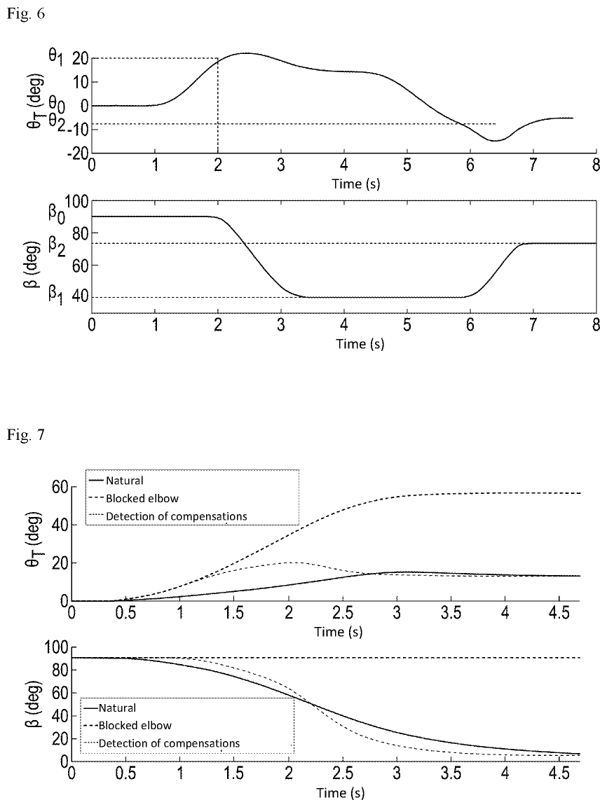

FIG. 6 is a graph illustrating the progression over time of the flexion angle of the trunk and the flexion angle of the elbow of the user in FIG. 2 to 4, according to a second embodiment, and

FIG. 7 is a graph illustrating different simulations of the progression over time of the flexion angle of the trunk and the flexion angle of the elbow in a user performing a pointing gesture and in different cases: natural gesture, gesture with blocked elbow (resulting in significant compensation of the trunk), and gesture with the proposed control mode (here automatically controlling the movement of the elbow according to the compensations of the trunk).

The device according to the invention makes it possible to control a robotic system for assisting mobility, and in particular for assisting the mobility of the upper limbs, for example a prosthesis or an exoskeleton of an upper limb. It uses as a control input the compensatory movements naturally implemented by the central nervous system (CNS) to perform functional tasks, such as, in the example which will be described, when the mobility of the upper limb(s) of the user is reduced.

These compensations, recorded by different sensors, allow the system to identify the movement, or motor intention, that the user wishes to perform and to deduce from it an action strategy of the robotic system, due in particular to an inverse kinematic model, for assisting the user in carrying out his task. The movement is then carried out by the robotic joint(s), while the user naturally and instinctively returns to a neutral posture, without postural compensation.

This is illustrated in FIG. 1. Θr denotes the reference position of the user's body, without compensation, while Θc denotes the current position of the body and 3′ denotes the matrix of angular speeds of the robotic joints.

In the example which will be described in reference to FIG. 2 to 7, the invention uses a control algorithm which makes it possible to assist the mobility of an upper limb. The algorithm allows intuitive control of the device because it is based on the use of strategies naturally implemented by the central nervous system to compensate for a reduction in said mobility. In a simplified version, which is the control of a single joint, that of the elbow, the device according to the invention was tested on ten healthy users whose movement of the elbow was driven by a robotic joint of the exoskeleton type. The users wore sensors on the trunk and on the arm. The experiment consisted of hitting a set of six targets, located at different distances and at different heights. All the users succeeded in the mobility action, correctly controlling the joint through the implementation of compensatory strategies, even though no explanation of the operation of the control of the robotic tool had been given to them.

Thus, as illustrated in FIG. 2, a user 1 is initially in a reference position, the trunk being arranged vertically. The reference position may be generally defined as being a comfortable position, without postural compensation, that the user would naturally adopt to perform the mobility action if he had control of all his joints. However, it is conceivable that the reference position of the trunk is a leaning position because certain gestures require the user to be naturally leaning. A non-static definition of the reference position may be added to the control law.

In the mobility action consisting in reaching a target at a distance from the user, the user whose arm is disabled will naturally tilt his trunk 5 forward to compensate for the disability of his arm. Thus, in the case where the mobility action is to reach a distant target 4, the associated compensatory movement is the forward inclination of the trunk 5. Alternatively, one could choose as the associated compensatory movement a movement of the shoulder or the shoulder blades.

When the user 1 leans forward (FIG. 3), sensors determine the inclination of the trunk 5 relative to the reference position in FIG. 2. It is possible to use sensors arranged on the trunk 5 of the user 1, such as deformable elastic sensors, or optical sensors.

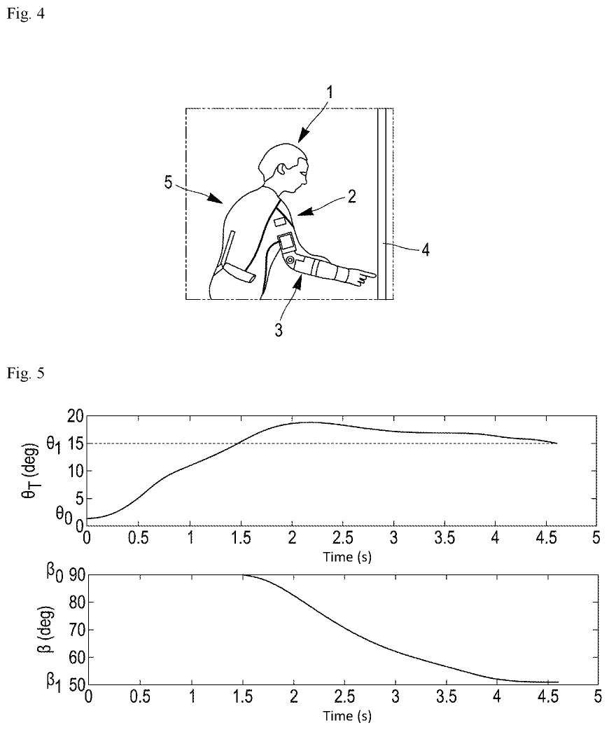

When the inclination of the trunk 5 reaches a predetermined value, the compensatory movement is detected, and the device 2 will control the active mobility assistance element, which in this example is a prosthetic elbow joint 3. When the compensatory movement is detected, the device 2 commands the prosthetic elbow to open, which will allow the user 1 to reach the target 4 (FIG. 4).

In this example, a two-dimensional inverse kinematic model is used for the elbow joint. However, a three-dimensional model may be considered more generally, with for example at least two motorized joints.

The invention is distinguished by its natural control law, which only uses the strategies already set up naturally by the central nervous system, and therefore requires very little learning. The user does not have to think about what to do. In addition, the control is carried out in the space of the mobility action (space of the task). What matters is not to move a joint, but to perform a given gesture, to position the hand in a given place. The output of the control law is not the movement of a single joint, but rather the coordinated movement of all the controlled joints necessary to perform the desired gesture, combined with the joints that the user always controls naturally (functional joint, stump, etc.). The joints are not controlled individually and sequentially, but simultaneously, as in a natural gesture. In the task of grasping an object, for example, no person without an amputated limb reflects on the individual position of his joints. The person is focused on the object to be reached, and therefore on the position of his hand, and he controls his arm in the space of the task.

The invention therefore reduces the learning time and the cognitive load. Its implementation is simple and fast because there is no training phase for the algorithm. The small number as well as the small size of the motion sensors allow for a use without constraint, in an open and home environment.

FIG. 5 illustrates the progression over time of the inclination angle of the trunk Θ_{T} and of the flexion angle of the elbow β of the user when the mobility action is the grasping of an object.

At the start of the process, the user's trunk is in its reference position Θ₀. The user, wishing to reach the targeted object with his hand, will initiate a compensatory movement which is tilting the trunk forward. When the inclination of the trunk reaches a predetermined value Θ₁, characteristic of the detection of the compensatory movement, the control system of the device commands the prosthetic elbow joint to open. The elbow thus opens progressively, from a flexion angle β₀ to a flexion angle β₁, which brings the hand closer to the object to be grasped.

FIG. 6 illustrates the progression over time of the inclination angle of the trunk OT and of the flexion angle of the elbow β of the user when the mobility action is the grasping of two objects.

Grasping the first object begins in the same way as in the embodiment shown in FIG. 5. From a reference position Θ₀ of the user's trunk, said user initiates a first forward compensatory movement of the trunk until the trunk is tilted to a predetermined value Θ₁, characteristic of the detection of the first compensatory movement. The device's control system commands the prosthetic elbow joint to open from a flexion angle β₀ to a flexion angle β₁, which brings the hand closer to the first object to be grasped.

Once the first object has been reached, the user then wishes to grasp a second object, which is closer to him than the first object. The user will then initiate a second compensatory movement, which is to tilt the trunk backwards, so as to bring his hand closer to the second object. When the inclination of the trunk reaches a predetermined value Θ₂, characteristic of the detection of the second compensatory movement, the control system of the device commands the prosthetic elbow joint to close. The elbow thus closes progressively, from the flexion angle β₁ to a flexion angle β₂ allowing the second object to be reached.

FIG. 7 illustrates different simulations of the progression over time of the inclination angle of the trunk and of the flexion angle of the elbow, the mobility action being to reach a target in space. Three configurations were considered: a user whose elbow joint is functional (“natural” curves), a user whose elbow joint is non-functional, without the assistance of the device (“blocked elbow” curves), and a user whose elbow joint is non-functional with the assistance of the device (“compensation detection” curves).

The simulations show that the behavior of the disabled user using the assistance device according to the invention, in terms of inclination of the trunk and flexion of the elbow, approaches the behavior of an able-bodied user.

The control system of the device according to the invention may also be seen as a simple approach to correcting the compensatory movement. In the example of grasping an object illustrated in FIG. 5, it may thus be considered that an active robotic element is in fact controlled so as to decrease the inclination of the torso when the compensatory movement has been detected: the opening of the elbow is actually increased so as to compensate for the decrease in the inclination of the torso.

In one embodiment of the invention, the device may not control a movement of the user toward the object to be reached, as just described, by moving the hand toward the object by opening or closing the elbow, but rather may control a movement of the object to be reached toward the user. In the example of grasping an object illustrated in FIG. 5, it is thus possible to envisage that an active robotic element integral with the object is controlled so as to bring the object closer to the hand of the user when the compensatory movement is detected. Naturally, this embodiment may be combined with the embodiment in which the user is controlled so as to move toward the object to be reached.

1. A device for controlling a robotic system for assisting the mobility of a user, said robotic system comprising at least one active mobility assistance element capable of assisting a given mobility action of the user, characterized in that the device comprises a detection system capable of detecting a compensatory movement of the user associated with the mobility action, said compensatory movement being a movement made by a user who is disabled, or able-bodied but locally and/or temporarily constrained, in order to perform at least part of the mobility action, and which at least partially substitutes the normal movement an unconstrained, able-bodied user would make in order to perform this mobility action, and a control system capable of controlling the at least one active element when the compensatory movement is detected.

2. The device according to claim 1, characterized in that the active element is a robotic arm prosthesis or an assistance arm exoskeleton.

3. The device according to claim 2, characterized in that the mobility action is to reach a target, and in that the compensatory movement is an inclination of the user's torso.

4. The device according to claim 1, characterized in that the detection system is configured to detect a difference between a reference position (Θr) and a position (Θc) of the user, and to detect a compensatory movement when said deviation reaches a predetermined deviation.

5. The device according to claim 1, characterized in that said at least one active mobility assistance element is an active element intended to be secured to the user.

6. The device according to claim 5, characterized in that said at least one active mobility assistance element is a motorized joint.

7. The device according to claim 1, characterized in that the active mobility assistance element is an active element external to the user.

8. The device according to claim 7, characterized in that the active mobility assistance element is an active element for controlling a movement of a target of the user.

9. The device according to claim 1, characterized in that the control system is further configured to induce a correction of the compensatory movement.

10. An assembly for assisting the mobility of a user, characterized in that it comprises a device according to claim 1 and a robotic system controlled by said device.

11. A method of controlling a robotic system for assisting the mobility of a user, characterized in that it implements a device according to claim 1.

12. The method according to claim 11, characterized in that it comprises the steps of:
determining a mobility action of a user,
associating said mobility action with a compensatory movement of the user,
when said compensatory movement is detected, controlling at least said active mobility assistance element.
